(19) United States
(12) Patent Application Publication
(10) Pub. No.: US 2018/0119171 A1
(43) Pub. Date: May 3, 2018

(54) REPLICATIVE VACCINIA VIRUS VECTOR HIV VACCINE

(71) Applicant: NATIONAL CENTER FOR AIDS/STD CONTROL AND PREVENTION, CHINESE CENTER FOR DISEASE CONTROL, Beijing (CN)

(72) Inventors: Yiming Shao, Beijing (CN); Qicheng ZHANG, Beijing (CN); Ying Liu, Beijing (CN)

(21) Appl. No.: 15/520,183

(22) Filed: Jan. 12, 2018

(30) Foreign Application Priority Data
Oct. 20, 2014 (CN) 201410558649.5

(51) Int. Cl. C12N 15/867; A61K 39/12; A61K 39/39; C07K 14/16; C12N 15/09; A61K 48/00; A61K 39/00; C12N 7/00

(52) U.S. Cl. CPC C12N 15/867; A61K 39/12; A61K 39/39; C07K 14/16; A61K 2039/53; A61K 48/00; A61K 39/00; C12N 7/00; C12N 15/09

(57) ABSTRACT

Provided is a recombinant replicative vaccinia virus, the virus comprising a polynucleotide encoding HSV-TK and HIV antigens. Also provided is a live vector HIV vaccine composition comprising the recombinant replicative vaccinia virus.

### TECHNICAL FIELD

The present invention relates to the field of medical biotechnology, and specifically relates to an AIDS vaccine of replicative vaccinia virus, which can induce a high level of humoral and cellular immune responses against human immunodeficiency virus and allow an effective control of severe adverse effects that might be caused by the vaccine.

### TECHNICAL BACKGROUND

Vaccinia virus vector is one of the most intensively and extensively studied viral vectors, which can be used as a vector for constructing vaccines. Non-replicative vaccinia virus vectors are safe, but have a relatively weaker immunogenicity. In particular, the immune response they induced in human is distinctly weaker than in monkey. Replicative vaccinia virus vectors have strong immunogenicity, and can induce long lasting immunity, but they have potential safety issues such as causing severe adverse effects.

Because smallpox had been eliminated for many years, vaccinia virus as a smallpox vaccine has no longer been inoculated for more than 30 years, and as a result, people rarely pay attentions to developing drugs against vaccinia virus. With the application of replicative vaccinia virus vectors in the field of vaccine development, it is desired to develop medicines and measures that can be used as effective treatments when severe adverse effects emerge.

### SUMMARY OF THE INVENTION

In a first aspect, the present invention provides a recombinant replicative vaccinia virus, in its TK region comprising a first polynucleotide encoding an HSV-TK and a second polynucleotide encoding an HIV antigen.

Preferably, the recombinant replicative vaccinia virus of the invention is a vaccinia virus Tiantan strain.

In one specific embodiment, the first polynucleotide in the recombinant replicative vaccinia virus of the invention comprises a nucleotide sequence set forth in SEQ ID NO: 3. In another embodiment, the first polynucleotide in the recombinant replicative vaccinia virus of the invention comprises a nucleotide sequence which is at least 80%, at least 85%, at least 90%, at least 95%, or at least 99% identical to the nucleotide sequence set forth in SEQ ID NO: 3, and wherein the first polynucleotide encodes a thymidine kinase (TK) from HSV-1.

In another specific embodiment, the second polynucleotide in the recombinant replicative vaccinia virus of the invention encodes a gp145 2M derived from 97CN001 which is the predominant HIV epidemic strain in China, and comprises a nucleotide sequence set forth in SEQ ID NO: 1. In another embodiment, the second polynucleotide in the recombinant replicative vaccinia virus of the invention encodes a gp145 2M derived from 97CN001, which is the predominant HIV epidemic strain in China, or an immunogenic fragment thereof. In another embodiment, the second polynucleotide in the recombinant replicative vaccinia virus of the invention comprises a nucleotide sequence which is at least 80%, at least 85%, at least 90%, at least 95%/6, or at least 99% identical to the nucleotide sequence set forth in SEQ ID NO:1, and wherein the second polynucleotide encodes a gp145 2M from HIV-1.

Preferably, the recombinant replicative vaccinia virus of the invention also comprises a third polynucleotide encoding an additional HIV antigen. In one preferred embodiment, the third polynucleotide is inserted into the HA region of the replicative vaccinia virus. In one specific embodiment, the third polynucleotide encodes a gag derived from 97CN001, which is the predominant HIV epidemic strain in China, and comprises a nucleotide sequence set forth in SEQ ID NO:2. In another embodiment, the third polynucleotide encodes a gag derived from 97CN001 which is the predominant HIV epidemic strain in China, or an immunogenic fragment thereof. In another embodiment, the third polynucleotide comprises a nucleotide sequence which is at least 80%, at least 85%, at least 90%, at least 95%, or at least 99% identical to the nucleotide sequence set forth in SEQ ID NO:2, and the third polynucleotide encodes a gag from HIV-1.

In one preferred embodiment, the present invention provides a recombinant replicative vaccinia virus, which is a vaccinia virus Tiantan strain comprising a gene encoding gp145 2M derived from 97CN001 which is the predominant HIV epidemic strain in China, and a gene encoding an HSV-TK, both genes being inserted into the TK region of the vaccinia virus Tiantan strain, and further comprising a gene encoding a gag derived from 97CN001 inserted into the HA region of the vaccinia virus Tiantan strain.

The invention further provides a composition of a live vector HIV vaccine, which comprises the recombinant replicative vaccinia virus of the invention mentioned in the first aspect and a pharmaceutically acceptable carrier and/or adjuvant.

The present invention also relates to a method for preventing and/or treating HIV infection in a subject, which comprises administrating the composition of the live vector HIV vaccine comprising the recombinant replicative vaccinia virus of the invention mentioned in the first aspect to the subject. The present invention also relates to the use of the recombinant replicative vaccinia virus of the invention mentioned in the first aspect in the preparation of a composition of a live vector HIV vaccine for preventing and/or treating HIV infection in a subject. Preferably, said subject is an immunocompromised patient, such as one having congenital immunodeficiency, HIV infection, or is undergoing immunosuppressive therapy.

The present invention also provides an AIDS immunization kit, and said kit comprises a plurality of components and an instruction indicating an immunization procedure, wherein one of the components is the composition of the live vector vaccine of the invention.

### DESCRIPTION OF THE DRAWINGS

FIG. 1 depicts the in vitro inhibition effects of GCV against the recombinant vaccinia virus.

FIG. 2 depicts the inhibition effects of GCV against TT-TK and Tr-EnvTK replications in the brains of mice.

FIG. 3 depicts the inhibition effects of GCV against TT-TK replication in the ovaries of mice.

FIG. 4 depicts immune responses in mice induced by TT-EnvTK; A: cellular immunity; B: humoral immunity.

FIG. 5 depicts the immune interference among several HIV antigens.

### DEPOSITION INFORMATION

The recombinant vaccinia virus TT-TK was deposited on Oct. 20, 2014, in China General Microbiological Culture Collection Center (CGMCC), under the deposition No.: CGMCC No. 9810.

The recombinant vaccinia virus TT-EnvTK was deposited on Oct. 20, 2014, in China General Microbiological Culture Collection Center (CGMCC), under the deposition No.: CGMCC No. 9808.

The recombinant vaccinia virus T-gag was deposited on Oct. 20, 2014, in China General Microbiological Culture Collection Center (CGMCC), under the deposition No.: CGMCC No. 9809.

### SEQUENCE INFORMATION

SEQ ID NO: 1: Gp145 2M gene sequence, which is originated from HIV-1;

SEQ ID NO:2: Gag gene sequence, which is originated from HIV-1;

SEQ ID NO:3: HSV-TK gene sequence, which is originated from HSV-1:

SEQ ID NO:4: plasmid pSC65-DR-GN gene sequence.

### DETAILED DESCRIPTION OF THE INVENTION

Acquired Immunodeficiency Syndrome, (AIDS) is a transmissible disease caused by an infection of Human Immunodeficiency Virus (HIV). Since the first case discovered in 1981, AIDS has been spread worldwide at an alarming rate, and becomes one of the most serious viral diseases threatening the human lives and health. The epidemicity of AIDS brings huge influences to the social and economic developments of the world. In some developing countries, HIV infections have resulted in shortenings of average lifespan, reductions of labor, and food shortages, which causing regressions in social and economic developments for 20 years. The epidemicity of HIV in China went through three stages: importing stage (1985-1988), spreading stage (1989-1994), and growing stage (1995-now).

In recent years, people gained great progresses in the researches and applications of anti-HIV medicines, and some of the infected patients received relatively good treatments. However, the emergence of medicine resistant strains causes the medicine not effective for every patient, the complicated administration procedure makes some of the patients unable to keep on taking medicines on time and thus result in failures of treatments, and expensive costs also make the treatments difficult to be widely popularized. The historical experiences demonstrated that the most cost-effective way to control the epidemicity of a disease is to use vaccines, successful examples including smallpox, poliomyelitis, measles, hepatitis, and so on. Accordingly, the development for a safe and effective AIDS vaccine is always the objective of scientific researchers.

The scientists in various countries all agree that an effective AIDS vaccine must be able to induce the generation of HIV specific CD8+ cytotoxic T-lymphocyte responses (CTL) and neutralizing antibody responses in an organism. It is difficult for current single vaccines to achieve such an objective, and a combined immunization using different types of vaccines is required, which will increase the immune-effects of the AIDS vaccines. Candidate vaccines can be chosen from the followings: traditional vaccines (inactivated vaccine and attenuated live vaccine), synthetic peptide and protein subunit vaccine, DNA vaccine, and live vector vaccine.

Compared to other types of vaccines, the advantages of live vector vaccines are in that: (1) they can actively infect target tissues or cells, and thus increase the efficiency of exogenous genes entering the cells; (2) vectors per se possesses an adjuvant effect, and can induce the generation of cytokines and chemokines; (3) most of them can induce immune responses for a long period of time.

Researches on using vaccinia virus as vectors for live vector vaccines are most profound and extensive, but most of vaccines entered clinical trial phase use non-replicative vectors, such as MVA, NYVAC, and ALVAC. However, the clinical trials show that the immunogenicity of non-replicative vectors is relatively weak, and the same vaccine can only induce distinctly weaker immune responses in human than in monkey. This might be related to the non-replicative feature thereof. After infecting host cells, non-replicative vector vaccine can only undergo one cycle of antigen expression, processing and presenting procedures, due to the fact that it cannot generate infectious virus and initiate a new cycle of infection, and thus the stimulation for immune system is only limited and short-acting. For this reason, the international researching focus for live vector vaccines has turned from non-replicative types to replicative types, so as to fully exert the replicative property of live vectors, and better stimulate the organisms to generate immune responses.

The replicative vaccinia virus vector has strong immunogenicity, and can induce long lasting immunity, but it has potential safety issues such as causing severe adverse effects. Especially for those individuals who are immunocompromized due to congenital immunodeficiency, immunosuppressive therapy, HIV infection, or so on, replicative vaccinia virus vector vaccines might trigger severe adverse effects^{[1,2]}. Because smallpox has been eliminated for many years, vaccinia virus as a smallpox vaccine has no longer been inoculated for more than 30 years, and thus people rarely pay attentions to developing drugs against vaccinia virus. With the application of replicative vaccinia virus vector in the field of vaccine development, it is desired to develop medicines and measures that can be used as effective treatments when severe adverse effects emerge.

Suicide gene system is a method widely used for treating diseases like cancer in recent years. Conventional combinations of suicide gene/medicine include: cytosine deaminase (CD)/5-fluorocytosine^{[3]}, nitroreductase/CB 1954^{[4]}, thymidine phosphorylase (TP)/5′-deoxy-5-fluorouridine (5′-DFUR)^{[5]}, purine nucleoside phosphorylase (PNP)/6-methylpurine-2′-deoxyriboside (MeP-dR)^{[6]}, and herpes simplex virus thymidine kinase (HSV-TK)/ganciclovir (GCV)^{[7]}, wherein HSV-TK/GCV is the suicide gene system that mostly researched and applied.

GCV activity is specifically depends on the TK gene of the HSV. The HSV TK can phosphorylate GCV, and generate a monophosphorylated form thereof (GCV-MP)^{[8]}. Subsequently cell kinase further phosphorylates the GCV-MP, and generates a diphosphate form (GCV-DP) and a toxic triphosphate form (GCV-TP). GCV-TP is a DNA polymerase inhibitor, which can inhibit the replication of DNA by competitively inhibiting the combination of deoxyguanosine triphosphate and DNA polymerase, or by inserting into a newly-generated chain to inhibit DNA replication, and resulted in a termination of the extension of the newly-generated chain, and ultimately kill the cells^{[9]}. Since GCV cannot be directly phosphorylated by the mammalian cell kinase, the prodrug only has selective toxicity against cells expressing the HSV-TK, with minor adverse effects.

Currently it has been reported that viral vectors carrying an HSV-TK/GCV suicide gene system are used for treating tumors, wherein the function of the HSV-TK/GCV suicide gene system is to directly kill the target cells, rather than to ensure the usage safety of the live virus vectors during the application procedure and control the possible adverse effects related to the vaccine vectors. Also, current HSV-TK/GCV suicide gene system is mainly used for adenovirus vectors and adeno-associated virus vectors, and the compatibility between the HSV-TK/GCV system and the vaccinia virus vector, especially the vaccinia virus Tiantan strain as the replicative vaccinia virus vector, has not yet been tested. The TK gene of vaccinia virus only has about 16% of homology to HSV-TK, and it seems that Ganciclovir has no inhibition effect on vaccinia virus (EC50>300 μM)^{[10]}.

The inventors introduced the HSV-TK gene into a replicative vaccinia virus vector, so as to provide a safe and controllable replicative vaccinia virus vector AIDS vaccine. The inventors demonstrated that the introduction of HSV-TK gene into the replicative vaccinia virus AIDS vaccine effectively controlled severe adverse effects that might be caused by the replicative vaccinia virus vector, increased the safety of the vaccine, and solved the safety issue caused by the replicative vaccinia virus vector without affecting the immunogenicity of the replicative poxvirus vector. The replicative vaccinia virus vector AIDS vaccine of the invention is particularly suitable for immunocompromised patients, such as patients having congenital immunodeficiency, HIV infection, or patients undergoing immunosuppressive therapies.

With respect to AIDS vaccines, the selection of HIV antigens is also very important. The HIV-specific function of eliminating CD8+ T-cells (CTL) plays an important role in HIV replications. Gag-specific CTL is advantageous for controlling virus replications in infected patients. Env-induced neutralizing antibody can neutralize HIV. The titre of V1V2 binding antibody is positively correlated with the immune protection of the vaccine. Therefore several antigens should be selected for an AIDS vaccine to induce different types of immune responses. Moreover, the inventors discovered that, with the prerequisite that the expression of several antigens is assured, the respective introductions of HIV-1 gag gene and gp145 2M gene into the HA region and the TK region of the replicative vaccinia virus vector can also avoid the interference between different antigens.

Accordingly, in a first aspect, the invention provides a recombinant replicative vaccinia virus comprising, in its TK region, a first polynucleotide encoding HSV-TK and a second polynucleotide encoding HIV antigen.

Preferably, the recombinant replicative vaccinia virus of the invention is a vaccinia virus Tiantan strain.

In one specific embodiment, the first polynucleotide in the recombinant replicative vaccinia virus of the invention comprises the nucleotide sequence set forth in SEQ ID NO:3. In another embodiment, the first polynucleotide in the recombinant replicative vaccinia virus of the invention comprises a nucleotide sequence which is at least 80%, at least 85%, at least 90%, at least 95%, or at least 99% identical to the nucleotide sequence set forth in SEQ ID NO:3, and the first polynucleotide encodes a TK from HIV-1.

In another specific embodiment, the second polynucleotide in the recombinant replicative vaccinia virus of the invention encodes a gp145 2M derived from 97CN001, the predominant HIV epidemic strain in China, and comprises the nucleotide sequence set forth in SEQ ID NO:1. In another embodiment, the second polynucleotide in the recombinant replicative vaccinia virus of the invention encodes a gp145 2M derived from 97CN001, the predominant HIV epidemic strain in China, or an immunogenic fragment thereof. In another embodiment, the second polynucleotide in the recombinant replicative vaccinia virus of the invention comprises a nucleotide sequence which is at least 80%, at least 85%, at least 90%, at least 95%, or at least 99% identical to the nucleotide sequence set forth in SEQ ID NO:1, and the second polynucleotide encodes a gp145 2M from HIV-1.

Preferably, the recombinant replicative vaccinia virus of the invention further comprises a third polynucleotide encoding an additional HIV antigen. In one preferred embodiment, the third polynucleotide is inserted into the HA region of the replicative vaccinia virus. In one specific embodiment, the third polynucleotide encodes a gag derived from 97CN001, the predominant HIV epidemic strain in China, and comprises the nucleotide sequence set forth in SEQ ID NO:2. In another embodiment, the third polynucleotide encodes a gag derived from 97CN001, the predominant HIV epidemic strain in China, or an immunogenic fragment thereof. In another embodiment, the third polynucleotide comprises a nucleotide sequence which is at least 80%, at least 85%, at least 90%, at least 95%, or at least 99% identical to the nucleotide sequence set forth in SEQ ID NO:2, and the third polynucleotide encodes a gag from HIV-1.

In one preferred embodiment, the invention provides a recombinant replicative vaccinia virus, which is a vaccinia virus Tiantan strain which comprises a gene encoding a gp145 2M derived from 97CN001, the predominant HIV epidemic strain in China, and a gene encoding HSV-TK, both being inserted into the TK region of the vaccinia virus Tiantan strain, and further comprises a gene encoding a gag derived from 97 CN001 inserted into the HA region of the vaccinia virus Tiantan strain.

The invention further provides a composition of live vector HIV vaccine, which comprises the recombinant replicative vaccinia virus of the invention described in the first aspect, and a pharmaceutically acceptable carrier and/or adjuvant.

The present invention also relates to a method for preventing and/or treating HIV infection in a subject, which comprises administrating to the subject the composition of live vector HIV vaccine that comprises the recombinant replicative vaccinia virus of the invention mentioned in the first aspect. The present invention also relates to use of the recombinant replicative vaccinia virus of the invention mentioned in the first aspect in the preparation of a composition of live vector HIV vaccine for preventing and/or treating HIA infections in a subject. Preferably, said subject is an immunocompromised patient, such as having congenital immunodeficiency, HIV infection, or undergoing immunosuppressive therapies.

The present invention also provides an AIDS immunization kit, which comprises a plurality of components and an instruction indicating the immunization procedure, wherein one of the components is the composition of the live vector vaccine of the invention.

### Examples

### Example 1: The Construction of Transfer Plasmids for the Recombinant Vaccinia Virus that Expresses HSV-TK Gene

The process for constructing the transfer plasmids pVI75-GFP-TK and pVI75-GFP-EnvTK is as following:

1. The Construction of Plasmid pSC65-TK1

The genomic DNA of herpes simplex virus HSV-1 was used as the template, primers HSV-TK-EcoRI/XhoI-up

(5' CAA [symbol] TCAGTGGTGGTGGTGGTGGTG
GTTAGCCTCCCCC 3', SEQ ID NO: 10)
and
HSV-TK-SalI-for
(5' TAC [symbol] ATGGCTTCGTACCCCTGCCATCAGC 3',
SEQ ID NO: 11)

carrying XbaI and SalI restriction sites and 3′-end His-tag were used for amplifying HSV-TK gene by PCR. The amplification conditions were as follows: 95° C. 5 min; 95° C. 30 s, 55° C. 30 s, 72° C. 1.5 min, 5 cycles; 95° C. 30 s, 60° C. 30 s, 72° C. 1.5 min. 25 cycles; 72° C. 5 min. The size of the HSV-TK fragment is 1149 bp.

The HSV-TK fragment obtained through PCR amplification was double-digested by enzymes SaI and EcoRI and then ligated into the plasmid pSC65 double-digested by the same enzymes (deposited on Feb. 24, 2004, in China General Microbiological Culture Collection Center (CGMCC), under the deposition No. CGMCC No. 1097), and obtained the pSC65-TK1 plasmid. The pSC65-TK1 plasmid was identified by SalI and EcoRI double-digestion resulting two fragments of 1351 bp and 4051 bp. The TK gene was sequenced, and the result was shown in SEQ ID NO:3.

2. The Construction of the Plasmid pSC65-TK1-Gp145 2M

In order to construct the recombinant plasmid pSC65-TK1-gp145 2M that simultaneously expresses HSV-TK and HIV-1gp145 2M, the plasmid pDRVISV1452M (the E. coli containing the plasmid was deposited on May 22, 2008, in CGMCC, under the deposition No. CGMCC No. 2513) was used as the template, and primers gp145 2M for-Sal (5′ ACGCGTCGACAGAGATATCGACACCATGG ACAGGG 3′, SEQ ID NO:12) and gp145 2M-rev-PacI (5′ GCCTTAATTAATCAGTAGCCCTGCCTCACCCTGTTC 3′, SEQ ID NO:13) were used to amplify the HIV-1 gp145 2M gene of 2.1 kb in size by PCR. The conditions for the PCR reaction were: 95° C. pre-denaturation for 5 min; 95° C. 30 s, 60° C. 30 s, 72° C. 2.5 min, altogether 30 cycles; 72° C. sufficient extension for 5 min. The HIV-1 gp145 2M fragment obtained by the amplification and the plasmid pSC65-TK1 were double-digested by SalI and PacI and then ligated to obtain the plasmid pSC65-TK1-gp145 2M. Identification was performed by double-digestion with PacI and XhoI resulting two fragments of 4076 bp and 3310 bp.

3. The Construction of the Transfer Plasmids pTK-gp1452M and pTK

The plasmids pSC65-TK1-gp145 2M and pSC65-DR-GN (the sequence of the plasmids can be seen in SEQ ID NO:4) were double-digested with PacI and XhoI and then ligated, resulting the transfer plasmid pTK-gp1452M. Double-digestion of the transfer plasmid pTK-gp1452M with PacI and XhoI results two fragments of 3461 bp and 6231 bp.

The plasmids pSC65-TK1 and pSC65-DR-GN were double-digested with PacI and XhoI and then ligated, resulting the transfer plasmid pTK. Primers HSV-TK-EcoRI/XhoI-up (SEQ ID NO:10) and HSV-TK-SalI-for (SEQ ID NO: 11) were adopted for PCR amplification, and the product was a fragment of about 1.2 kb in size.

### Example 2: Construction and Characterization of the Recombinant Vaccinia Virus

The TKL and TKR regions of the transfer plasmids pTK and pTK-gp1452M were homologously recombined with the vaccinia virus Tiantan strain, allowing the target genes HSV-TK and GP145 2M together with marker genes neo and GFP to be recombined into the TK sequence of the vaccinia virus genome. In the presence of G418, homologous recombination did not take place within the molecules due to the selective pressure, marker genes neo and GFP temporally remained in the genome. The recombinant vaccinia virus that not only contains the target gene but also possesses green fluorescence was selected under fluorescence microscope (the growth of virus in which no recombination took place was inhibited due to the presence of G418). Subsequent selection was conducted under the conditions without G418, the recombinant vaccinia virus with green fluorescence per se can undergo an intramolecular secondary homologous recombination between an intact TKR and a small section of about 200 bp of a TKR-DR fragment brought in by the transfer plasmid. Therefore, the neo gene and GFP gene are lost. The recombinant vaccinia virus that only contains the target gene was obtained by selection of recombinant virus without green fluorescence.

1. Construction and Purification of the Recombinant Vaccinia Virus

Chicken embryo fibroblasts (CEFs) of 80-90% confluency were infected with a vaccinia virus Tiantan strain VTT (the virus was provided by National Vaccine and Serum Institute of Beijing) at a viral exposure amount of MOI=1, and cultivated at 37° C., 5% CO₂ for 2 hours. Using liposome to respectively transfect the recombinant plasmids pTK and pTK-gp145 2M into the cells, and the process is delineated in the instruction manual of the kit (Invitrogen, Lipofectamine® 2000 transfection Reagent. Cat. 11668-027). The recombinant plasmids underwent homologous recombination with VTT in the cells, and simultaneously resulted in losses of the VTT thymidine kinase gene. 24 hours after the transfection, the cell sample was collected and frozen-thawed for 3 times.

CEF cells in confluency was first cultured using the Eagle's medium containing 0.4 mg/mL of G418 for 24 h, 300 μl of the collected transfection virus liquid was inoculated to CEF cells pre-treated by 0.4 mg/mL of G418 for 24 h. The Eagle's medium still contained 0.4 mg/mL of G418 and 1% of low melting-point agarose. After being cultured at 37° C. for 48 hours, a plaque with green fluorescence was picked and inoculated into 1 mL of the Eagle's maintenance medium. After being frozen-thawed for 3 times, 100 μl of the medium was inoculated to CEF cells that have been pre-treated by G418. By repeating the above procedures, single plaques were continuously purified for 2 generations, and the third generation of plaque with green fluorescence was thus obtained. The virus picked at this point contained gp145 2M, TK, GFP and neo genes. After frozen-thawing the third generation of plaque with green fluorescence for 3 times, 100 μl of each sample was respectively picked to infect CEF cells without G418 treatment. The cells were cultivated at 37° C. for 48 hours, and then white plaques without fluorescence were selected. After freeze-thawing the white plaque virus for 3 times, 100 μl of sample was then picked to inoculate CEF cells pre-treated by G418. By repeating the above procedure and continuously purifying single plaques for 5 generations, the purified recombinant vaccinia viruses TT-TK and TT-EnvTK were obtained. TT-TK and TT-EnvTK were deposited on Oct. 20, 2014, in China General Microbiological Culture Collection Center (CGMCC), under the deposition No.: TT-TK: CGMCC No. 9810, TT-EnvTK: CGMCC No. 9808, respectively.

2. PCR Identification of the Recombinant Virus

The genomic DNA of recombinant viruses TT-TK and TT-EnvTK were extracted, and identification primers tkL-up (TAACGTGATGGATATATTAAAGTCG, SEQ ID NO:14) and tkR-low (AACGACACAACATCCATITITAAG, SEQ ID NO:15) were used to perform a PCR identification. The results showed that the sizes of the fragments using VTT, TT-TK, and TT-EnvTK as templates were 674 bp, 2830 bp, and 4966 bp respectively, indicating that both gp145 2M and TK genes were correctly recombined into the VTT genome.

3. Expression of Target Gene of the Recombinant Vaccinia Virus

The recombinant vaccinia viruses TT-TK. TT-EnvTK, VTT, and VTKgpe (deposited in China General Microbiological Culture Collection Center, under the deposition No.: CGMCC No. 1099) were respectively used at a dose of 1 PFU/cell to infect CEF cells. Cells were collected after 24 hours cultivation, 1 ml of protein extraction buffer (1% SDS, 1 mmol/L PMSF, 20 mmol/L Tris-Cl pH7.0, 1% β-mercaptoethanol) was added, and immediately blended, and repeatedly frozen-thawed for 3 times. PAGE electrophoresis and membrane transfer were performed, and 5% skim milk was used for blocking at room temperature for 2 hours. Mouse anti-His-Tag antibody (Gibco, Cat. Fk0168) was used to detect the expression of HSV-TK, and HIV-1 SF2 GP160 (from NIH AIDS Research & Reference Reagent Program) anti-serum was used to detect the expression of HIV-Env (1:1000 diluted). The membranes were incubated with primary antibodies diluted in 5% skim milk at the room temperature for 2 hours, and washed with PBS for 3 times, 10 minutes for each time. Corresponding secondary antibodies labeled by horseradish peroxidase diluted at 1:5000 in 5% skim milk were added and incubated at the room temperature for 1 hour. The membranes were washed with PBS for 3 times, 10 min for each time. After ECL color development, the results showed that, TT-TK and TT-EnvTK had a band of about 40 kD, which was the same size as HSV-TK-His, indicating that the recombinant virus strain TT-TK can express HSV-TK gene. An HIV-Env band of about 140 kD was detected in both TT-EnvTK and the positive control, indicating that the recombinant virus strain TT-EnvTK can express the HIV-Env gene.

### Example 3: The Inhibition Effect of Ganciclovir (GCV) Against the Recombinant Vaccinia Virus

1. The In Vitro Inhibition Experiment of GCV for the Recombinant Vaccinia Viruses

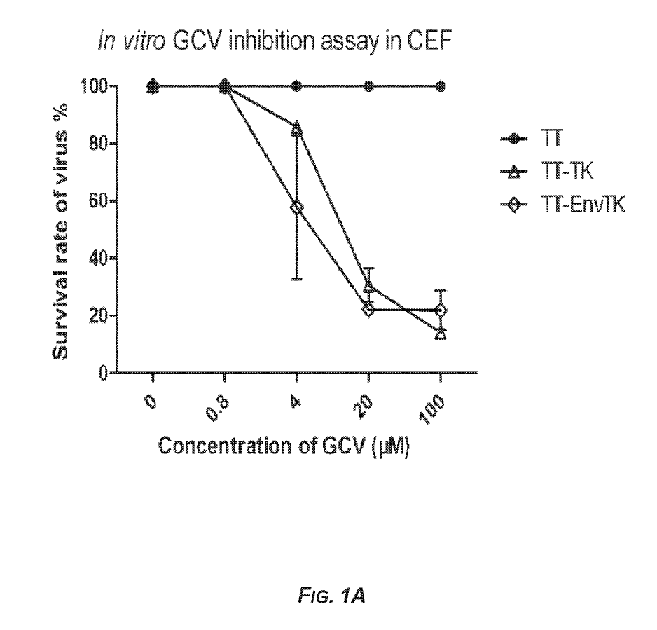

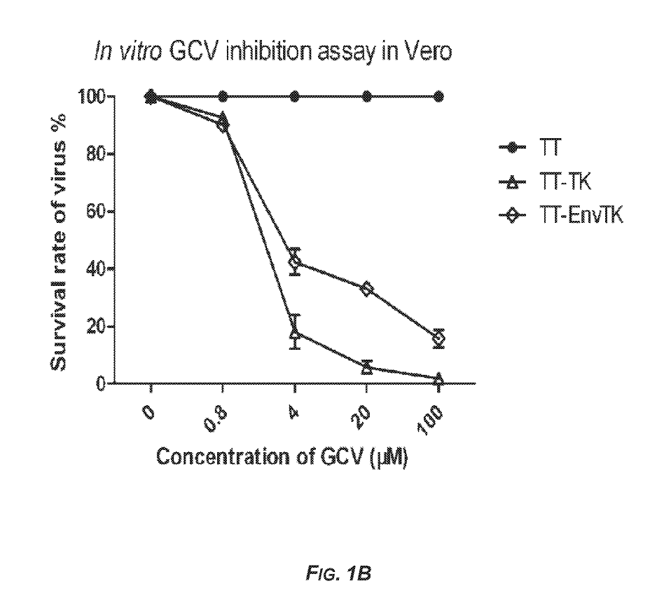

VTT, TT-TK, and TT-TKEnv were respectively inoculated at a virus exposure amount of about 100 PFU/well to infect a single layer of CEF or Vero cells in a 6-well plate. After infection at 37° C., 5% CO₂ for 2 hours, a medium containing a final concentration of 0˜100 μM of GCV was then added to the cells. After continuously cultivated for 48 hours, the number of plaques formed in each well was counted, and inhibition curves of GCV against each of the recombinant vaccinia viruses were plotted. The results are shown in FIG. 1. In CEF (A) and Vero (B), GCV showed clear inhibition effects on TT-TK and TT-EnvTK, and the virus plaque numbers for both of TT-TK and TT-EnvTK were negatively correlated to the concentration of GCV. GCV showed no inhibition effects on VTT.

2. The In Vivo Inhibition Experiment of GCV for the Recombinant Vaccinia Viruses

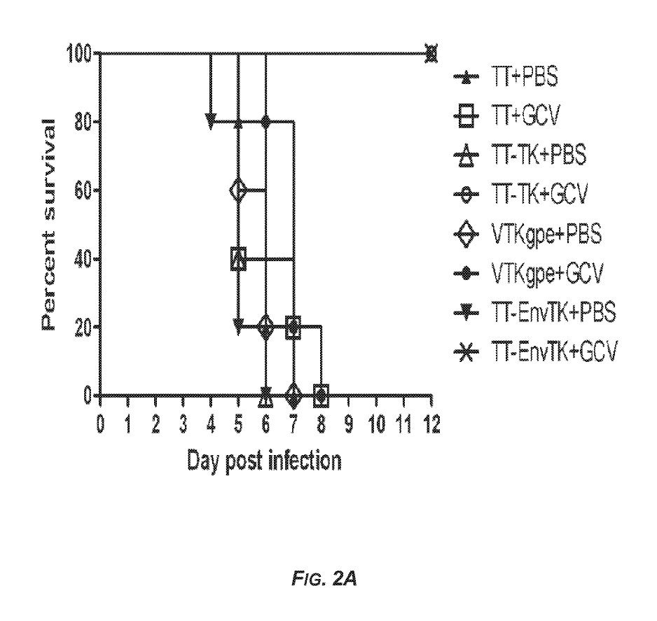

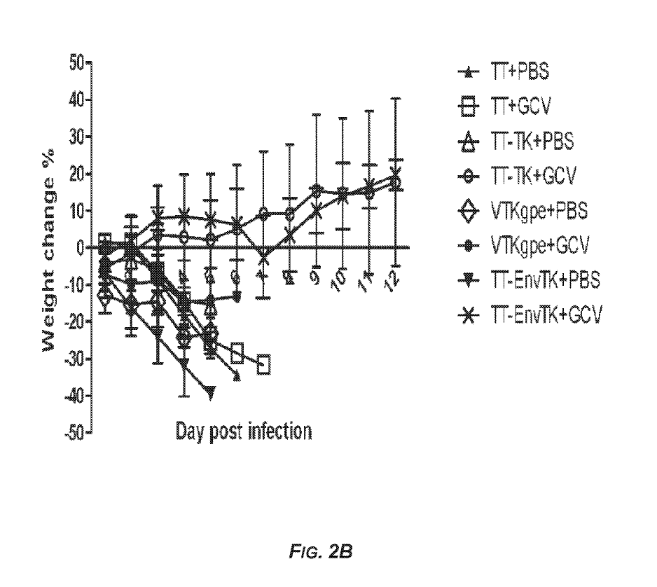

Three-week old BALB/c mice were randomly divided into different groups, which were inoculated in brain with 10×LD₅₀ doses of VTT, TT-T, or TT-EnvTK, and then were injected with a dose of 80 mg/kg/day of GCV. PBS was set as a control group. The changes of body weight and mortality of mice in each group were recorded every day. The results are showed in FIG. 2. During the experiment, the mice in PBS control groups began to show decreases in body weights and activities 3 days after the virus challenge. All the animals in each control group died on day 7. The body weight loss and the mortality of the VTT-mice (mice inoculated with VTT) in GCV injected group were similar to the PBS control group. All the animals died on day 8. The body weights of the TT-TK-mice and TT-EnvTK-mice (inoculated with TT-TK and TT-EnvTK) in GCV injected groups slightly decreased, and gradually recovered since day 3. All the animals survived. This result demonstrates that GCV can inhibit the replication of TT-TK and TT-EnvTK in the brain of mice.

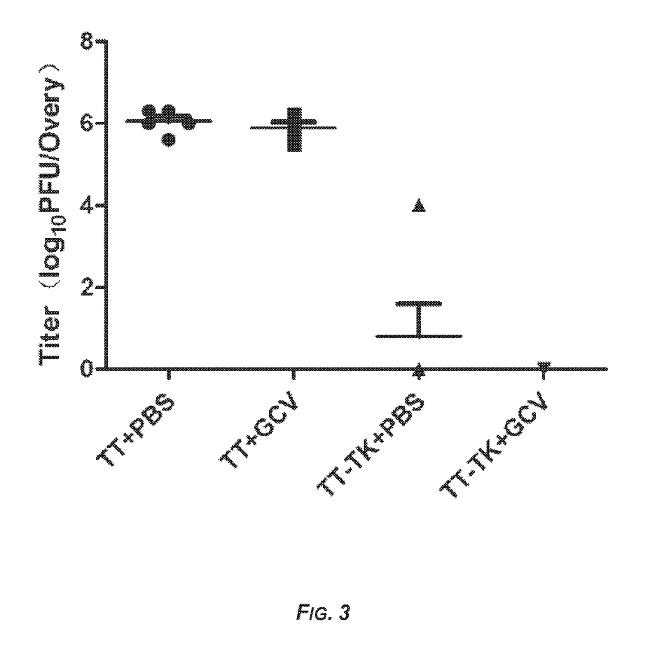

Eight-week old female C57BL mice were randomly divided into different groups. Each was injected in the abdominal cavity using 10⁷ PFU doses of VTT and TT-TK. Mice were treated with abdominal injection of GCV in a dose of 80 mg/kg/day and PBS was set as control groups. The mice in each group were sacrificed 8 days after the virus challenge and the ovaries on both sides were removed. The viral titre therein was titrated on CEFs. The results are shown in FIG. 3. Injected with PBS or GVC after VTT inoculation, the virus titres in mice ovaries were up to 1.2×10⁶ PFU and 1×10⁶ PFU, respectively. Injected with PBS after TT-TK inoculation, the virus titre in mice ovaries was 2×10³ PFU. No virus was detected in mice ovaries that were injected with GCV. This result indicated that GCV can inhibit the replication of TT-TK in mice ovary.

### Example 4: The Immunogenicity of the Recombinant Vaccinia Virus

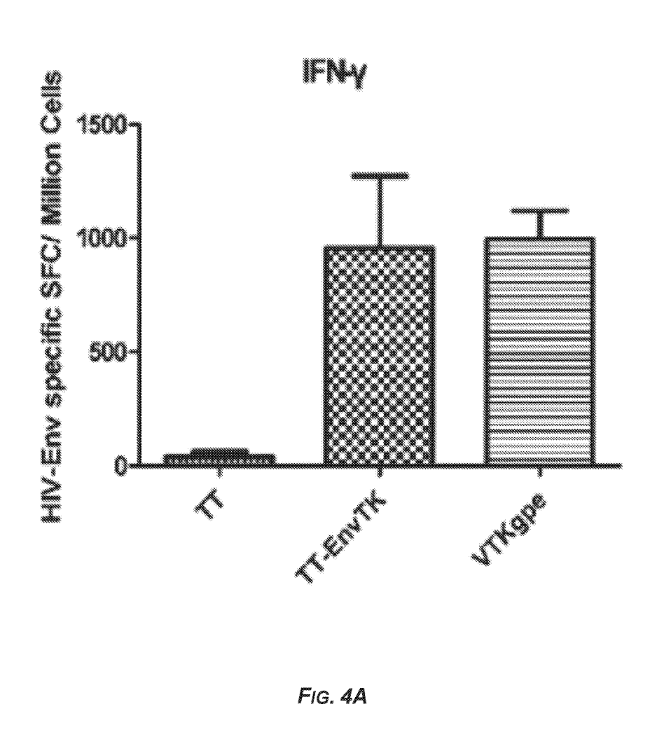

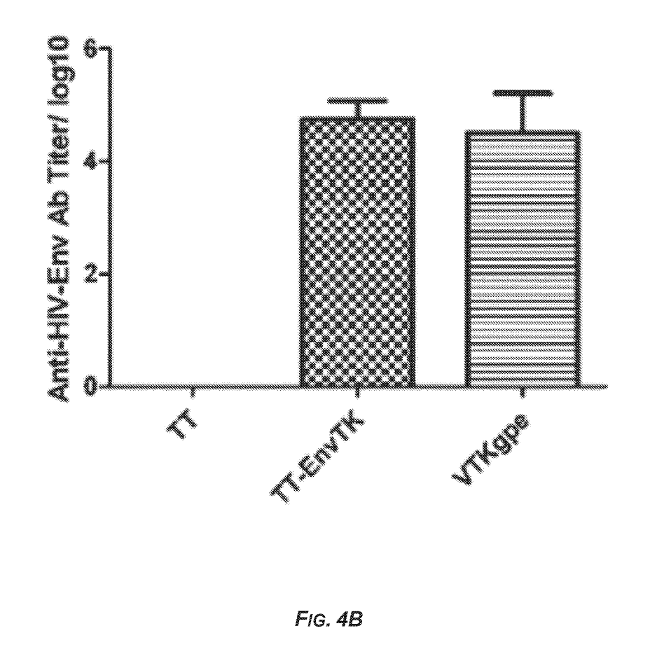

Six-week old female BALB/c mice were randomly divided into different groups, with 5 mice in each group. DNA vaccines of 50 μg/mouse/dose were intramuscularly injected at week 0, 3, and 6, and at week 9, 10⁷ PFU of TT-EnvTK, as well as control virus strains VTKgpe and VTT, were injected respectively as boosters. One week after the injections of the boosters, the mice were sacrificed, serum was isolated, and splenic lymphocytes were prepared. ELISA process was used to detect HIV-specific antibody, the ELISPOT of IFN-γ was used to detect cellular immune responses. The results showed that, the strength of the specific T-cell immune responses induced by the recombinant virus TT-EnvTK against HIV-Env is 956 SFC/10⁶ splenic cells, and that induced by the control vaccine strain VTKgpe is 996 SFC/10⁶ splenic cells. Statistical analysis showed that, there was no significant difference between the two groups (P>0.05) (FIG. 4A). The antibody titre induced by TT-EnvTK was 10^{4.7}, and that induced by VTKgpe was 10^{4.5}. Statistical analysis showed that, there was no significant difference between the two groups (P>0.05) (FIG. 4B). The results showed that, TT-EnvTK can induce HIV-specific cellular and humoral immune responses, and the strength of immune responses showed no significant difference with the control virus.

### Example 5: The Interference Among Several HIV Antigen Genes in the Same Recombination Region

The expression of several HIV antigen genes in the same recombination region can simplify the virus construction procedure, but there seemed to be certain interference among the exogenous genes. Regarding this, the inventors compared the gag specific cellular immune responses induced by recombinant viruses TT-gag and VTKgpe.

The TK region of VTKgpe simultaneously expressed gag, pol, and env genes of HIV, while the recombinant vaccinia virus TT-gag (deposited on Oct. 20, 2014, in China General Microbiological Culture Collection Center (CGMCC), under the deposition No.: CGMCC No. 9809) only expressed gag gene.

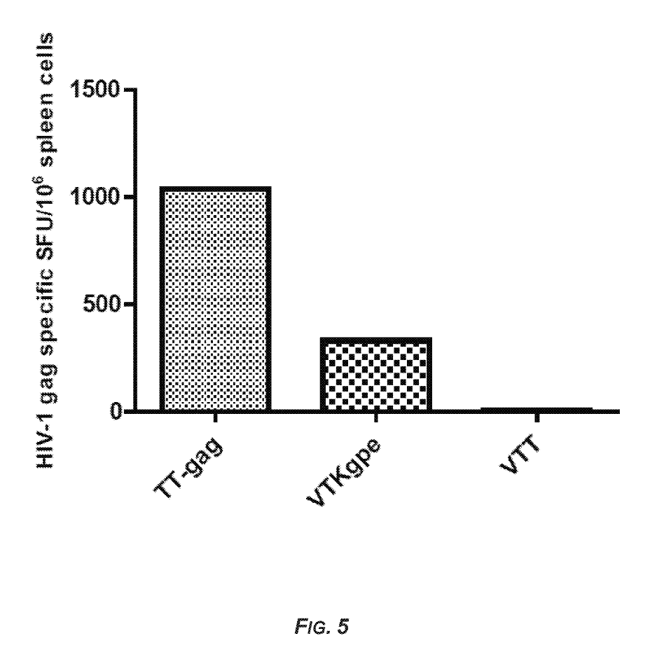

Six-week old female BALB/c mice were randomly divided into different groups, with 5 mice in each group. DNA vaccines of 50 g/mouse/dose were intramuscularly injected at week 0, 3, and 6, and at week 9, VTKgpe, TT-gag, and control virus VTT were injected as boosters respectively. One week after the injections of the boosters, the mice were sacrificed, serum was isolated, and splenic lymphocytes were prepared. The ELISPOT results of IFN-γ showed that, the strength of the specific T-cell immune responses against HIV-1 gag induced by the recombinant virus VTKgpe was 330 SFC/10⁶ splenic cells, which was significantly lower than the 1038 SFC/10⁶ splenic cells induced by TI-gag (FIG. 5). Therefore, according to the result, the interference among the several HIV antigen genes in the same recombination region indeed existed, which decreased the strength of immune responses. Therefore, HIVgag, gp145 2M genes should be inserted into different regions of the Tiantan strain respectively, such as HA region, to ensure that gag can induce stronger cellular immunity.

### Example 6: Multivalent HIV Vaccine that Expresses Different Antigens in Several Recombination Regions

1. Construction of Transfer Plasmid pVI76-Gag

Using the genomic DNA of vaccinia virus Tiantan strain VTT (the virus was provided by National Vaccine and Serum Institute of Beijing) as the template, primer A55R-for with EcoRI restriction site (5′ CATACGCGATCAGAATTCATCGTTGACATCTAGTATTGA TAG 3′, SEQ ID NO: 16) and primer A55R-Rev with StuI, XhoI and AscI restriction sites (5′ TAAGGCCTCTCGAGGCGCGCCCTATCAACTACCTATAAAACTTTCC 3′, SEQ ID NO: 17) were used to amplify A55R fragment by PCR. The amplification conditions were as follows: 95° C. 5 min; 95° C. 30 s, 55° C. 30 s, 72° C. 1 min, 30 cycles; 72° C. 5 min. The size of A55R fragment was 621 bp. Primer A57R-for with XhoII and PacI restriction sites (5′ GAGAACCTCGAGTTAATTAATGACTIACATAAATGTCTGGGATAG 3′, SEQ ID NO:18) and primer A57R-Rev with StuI restriction site (5′ TCTAGGCCTTGTTAAAA TACATTCTAATACGGTC 3′, SEQ ID NO:19) were used to amplify A57R fragment by PCR. The amplification conditions were as follows: 95° C. 5 min; 95° C. 30 s, 55° C. 30 s, 72° C. 1 min, 30 cycles: 72° C. 5 min. The size of A57R fragment was 600 bp. Primer A55R-DR-for with XhoII restriction site (5′ AAATCTCGAGAGAATTAATCCCG CTCTATGGTCAG 3′, SEQ ID NO:20) and primer A55R-DR-Rev with HindI restriction site (5′ GCGAAGCTTTTGTTCTATCAACTACCTATAAAAC 3′. SEQ ID NO:21) were used to amplify A55R-DR fragment by PCR. The amplification conditions were as follows: 95° C. 5 min; 95° C. 30 s, 55° C. 30 s, 72° C. 30 s, 30 cycles; 72° C. 2 min. The size of A55R-DR fragment was 279 bp.

The A55R fragment obtained by the PCR amplification was double digested by XhoI and EcoRI. The A57R fragment was double digested by XhoI and StuI. The digested fragments were then ligated into the plasmid pUC57 (Genscript, Cat. SD1176), which was double digested by XhoI and StuI. A pUC57-A55RA57R plasmid was obtained. The A55R and A57R fragments were sequenced, and the results were showed in SEQ ID NO:5 and SEQ ID NO:6, respectively.

The plasmid pLW73-Neo-I8RDR (plasmid sequence is shown in SEQ ID NO:7) was double digested by AscI and XhoI, and a fragment Neo-GFP was obtained. The fragment Neo-GFP was ligated into the plasmid pUC57-A55RA57R, which was digested by the same restriction enzymes. A transfer plasmid pUC57-A55RA57R-GN was obtained. The size of the plasmid was 5763 bp.

PCR amplified fragment A55R-DR was double digested by enzymes HindIII and XhoI, and was then cloned into a plasmid pSC65, which was digested by the same restriction enzymes. A recombinant plasmid pSC65-A55R-DR was obtained. The A55R-DR fragment was sequenced, and the result was shown in SEQ ID NO:8. The plasmid pSC65-A55R-DR was double digested by PacI and XhoI, and a fragment pE/L-DR was obtained. The fragment was ligated into the plasmid pUC57-A55RA57R-GN, which was double digested by PacI and XhoI. A plasmid pVI76 was obtained. The plasmid size was 6117 bp. The plasmid was sequenced, and the result was shown in SEQ ID NO:9.

The Gag fragment (SEQ ID NO:2) and the plasmid pVI76 were double digested by KpnI and PacI and then ligated. A transfer plasmid pVI76-gag was obtained. KpnI and PacI restriction enzymes were used for double digestion verification. The result showed that, the sizes of the enzyme digestion products were 1488 bp and 6117 bp respectively, indicating that Gag gene was correctly inserted into plasmid pVI76.

2. Construction and Purification of Recombinant Viruses

The recombinant virus TT-EnvTK (CGMCC No. 9808) was inoculated at a viral exposure amount of MOI=1 to infect chicken embryo fibroblasts (CEFs) of 80-90% confluency, which were then cultivated at 37° C., 5% CO₂ for 2 hours. Liposome was used to transfect the recombinant plasmid pVI76-gag into the cells, according to the process delineated in the instruction manual of the kit (Invitrogen, Lipofectamine® 2000 transfection Reagent, Cat. 11668-027). The recombinant plasmid underwent homologous recombination with TT-EnvTK in HA region in the cell. 24 hours after the transfection, the cell sample was collected and frozen-thawed for 3 times.

The selection process for recombinant viruses was the same as Example 2, the purified recombinant vaccinia virus TT-TK+/EG was obtained through 3 generations of green fluorescence plaques and 5 generations of white plaques selection purification.

3. PCR Identification of Recombinant Viruses

The genomic DNA of the recombinant virus TT-TK+/EG was extracted, and identification primers tkL-up (SEQ ID NO:14) and tkR-low (SEQ ID NO:15), SQEA57R-rev (5′ TGTTAAAATACATTCTAATACGGTC 3′, SEQ ID NO:22) and SQE55R-for (5′ ATCGTTGACATCTAGTATTGATAG 3′, SEQ ID NO:23) were used for PCR identification. The results showed that, the sizes of the fragments amplified by the two pairs of primers were 4966 bp and 3047 bp respectively, indicating that gp145 2M and HSV-TK were correctly recombined into the TK region of VTT genome, and Gag gene was correctly recombined into the HA region of VTT genome.

4. The Expression of the Target Gene in the Recombinant Vaccinia Virus

The 1-5# clones of the recombinant vaccinia virus TT-TK+/EG were respectively inoculated at a dose of 1 PFU/cell to infect CEF cells, and the cells were collected after being cultivated for 24 hours. 1 ml of protein extraction buffer (1% SDS, 1 mmol/L PMSF, 20 mmol/L Tris-Cl pH7.0, 1% β-mercaptoethanol) was added to the cells, and immediately blended, and repeatedly frozen-thawed for 3 times. PAGE electrophoresis and membrane transfer were performed, and 5% skim milk was used for blocking at the room temperature for 2 hours. HIV-1 SF2 GP160 anti-serum and HIV-1 Gag anti-serum (both from NIH AIDS Research & Reference Reagent Program) were used to respectively detect the expressions of HIV-1 Gp145 2M and Gag. A primary antibody diluted by 5% skim milk was incubated at the room temperature for 2 hours. PBS membrane was washed for 3 times, and each time for 10 min. A corresponding secondary antibody labeled by horseradish peroxidase was added, diluted at 1:5000 using 5% skim milk, and incubated at the room temperature for 1 hour. PBS membrane was washed for 3 times, and each time for 10 min. After ECL color development, the results showed that, 1-5# recombinant viruses can all be detected of the HIV-1Gp145 2M band of about 140 kD and the HIV-1 Gag band of about 55 kD, indicating that the recombinant viruses can correctly express the Gp145 2M and Gag genes.

### REFERENCES

1. Redfield R R, Wright D C, James W D, et al., Disseminated vaccinia in a military recruit with human immunodeficiency virus (HIV) disease. N Engl J Med, 1987. 316(11): 673-676.
2. Kesson A M, Ferguson J K. Rawlinson W D, et al., Progressive vaccinia treated with ribavirin and vaccinia immune globulin. Clin Infect Dis. 1997. 25(4): 911-914.
3. Austin E A and Huber B E, A first step in the development of gene therapy for colorectal carcinoma: cloning, sequencing, and expression of Escherichia coli cytosine deaminase. Mol Pharmacol. 1993. 43(3): 380-387.
4. Bridgewater J A, Springer C J, Knox R J, et al., Expression of the bacterial nitroreductase enzyme in mammalian cells renders them selectively sensitive to killing by the prodrug CB1954. Eur J Cancer, 1995. 31A(13-14): 2362-2370.
5. Patterson A V, Zhang H, Moghaddam A, et al., Increased sensitivity to the prodrug 5′-deoxy-5-fluorouridine and modulation of 5-fluoro-2′-deoxyuridine sensitivity in MCF-7 cells transfected with thymidine phosphorylase. Br J Cancer, 1995. 72(3): 669-675.
6. Parker W B, King S A, Allan P W, et al., In vivo gene therapy of cancer with E. coli purine nucleoside phosphorylase. Hum Gene Ther, 1997. 8(14): 1637-1644.
7. Kraiselburd E, Thymidine kinase gene transfer by herpes simplex virus. Bull Cancer, 1976. 63(3): 393-398.
8. Reardon J E, Herpes simplex virus type 1 and human DNA polymerase interactions with 2′-deoxyguanosine 5′-triphosphate analogues. Kinetics of incorporation into DNA and induction of inhibition. J Biol Chem, 1989. 264(32): 19039-19044.
9. Matthews T and Boehme R, Antiviral activity and mechanism of action of ganciclovir. Rev Infect Dis, 1988. 10 Suppl 3: S490-494.
10. Kern E R, In vitro activity of potential anti-poxvirus agents. Antiviral Res, 2003. 57(1-2): 35-40.

1. A recombinant replicative vaccinia virus, comprising, in its TK region, a first polynucleotide encoding an HSV-TK and a second polynucleotide encoding an HIV antigen.

2. The recombinant replicative vaccinia virus of claim 1, wherein the replicative vaccinia virus is a vaccinia virus Tiantan strain.

3. (canceled)

4. The recombinant replicative vaccinia virus of claim 1, wherein the first polynucleotide comprises a nucleotide sequence which is at least 80%, at least 85%, at least 90%, at least 95%, at least 99%, or which is 100% identical to the nucleotide sequence set forth in SEQ ID NO:3, and further wherein the first polynucleotide encodes a TK from HSV-1.

5. (canceled)

6. The recombinant replicative vaccinia virus of claim 1, wherein the second polynucleotide encodes a gp145 2M derived from 97CN001 which is the predominant HIV epidemic strain in China, or an immunogenic fragment thereof.

7. The recombinant replicative vaccinia virus of claim 1, wherein the second polynucleotide comprises a nucleotide sequence which is at least 80%, at least 85%, at least 90%, at least 95%, at least 99%, or which is 100% identical to a nucleotide sequence as set forth in SEQ ID NO:1, and further wherein the second polynucleotide encodes a gp145 2M from HIV-1.

8. The recombinant replicative vaccinia virus of claim 1, further comprising a third polynucleotide encoding an additional HIV antigen.

9. The recombinant replicative vaccinia virus of claim 8, wherein the third polynucleotide is inserted into the HA region.

10. (canceled)

11. The recombinant replicative vaccinia virus of claim 9, wherein the third polynucleotide encodes a gag derived from 97CN001 which is the predominant HIV epidemic strain in China, or an immunogenic fragment thereof.

12. The recombinant replicative vaccinia virus of claim 9, wherein the third polynucleotide comprises a nucleotide sequence which is at least 80%, at least 85%, at least 90%, at least 95%, at least 99%, or which is 100% identical to a nucleotide sequence set forth in SEQ ID NO:2, and wherein the third polynucleotide encodes a gag from HIV-1.

13. The recombinant replicative vaccinia virus of claim 2, wherein the recombination replicative vaccinia virus comprises a gene encoding a gp145 2M derived from 97CN001 which is the predominant HIV epidemic strain in China, and a gene encoding an HSV-TK, both genes being inserted into the TK region of the vaccinia virus Tiantan strain, and further comprises a gene encoding a gag derived from the 97CN001 inserted into the HA region of the vaccinia virus Tiantan strain.

14. A composition of a live vector HIV vaccine comprising the recombinant replicative vaccinia virus of claim 1 and a pharmaceutically acceptable carrier or adjuvant.

15. A method for treating or preventing HIV infection in a subject, the method comprising administering the recombinant replicative vaccinia virus of claim 1 to the subject.

16. The method of claim 15, wherein said subject is an immunocompromised patient.

17. The method of claim 16, wherein said subject has congenital immunodeficiency, has an HIV infection, or is undergoing an immunosuppressive therapy.

18. (canceled)


SEQUENCE LISTING (23 sequences)

SEQ ID NO: 1 - DNA, 2097 bp, HIV-1
atggacagggccaagctgctgctgctgctgctgctgctgctgctgccccaggcccaggccgtgggcaacctgtgggtgaccgtgtactacggcgtgcccgtgtggaagggcgccaccaccaccgagttctgcgccagcgacgccaaggcctacgacaccgaggtgcacaacgtgtgggccacccacgcctgcgtgcccgccgaccccaacccccaggagatggtgctggagaacgtgaccgagaacttcaacatgtggaagaacgagatggtgaaccagatgcaggaggacgtcatcagcctgtgggaccagagcctgaagccctgcgtgaagctgacccccctgtgcgtgaccctggagtgcaggaacgtgagcagcaaccaggacacctaccacgagacctaccacgagagcatgaaggagatgaagaactgcagcttcaacgccaccaccgtggtgagggacaggaagcagaccgtgtacgccctgttctacaggctggacatcgtgcccctgaccaagaagaactacagcgagaacagcagcgagtactacaggctgatcaactgcaacaccagcgccatcacccaggcctgccccaaggtgaccttcgaccccatccccatccactactgcacccccgccggctacgccatcctgaagtgcaacgacaagatcttcaacggcaccggcccctgccacaacgtgagcaccgtgcagtgcacccacggcatcaagcccgtggtgagcacccagctgctgctgaacggcagcctggccgagggcgagatcatcatcaggagcgagaacctgaccaacaacgtgaaaaccatcatcgtgcacctgaaccagagcgtggagatcgtgtgcaccaggcccggcaacaacaccaggaagagcatcaggatcggccccggccagaccttctacgccaccggcgacatcatcggcgacatcaggcaggcccactgcaacatcagcgaggacaagtggaacgagaccctgcagagggtgagcaagaagcttgccgagcacttccagaacaagaccatcaagttcgccagcagcagcggcggcgacctggaggtgaccacccacagcttcaactgcaggggcgagttcttctactgcaacaccagcggcctgttcaacggcgcctacacccccaacggcaccaagagcaacagcagcagcatcatcaccatcccctgcaggatcaagcagatcatcaacatgtggcaggaggtgggcagggccatgtacgcccctcccatcaagggcaacatcacctgcaagagcaacatcaccggcctgctgctggtgagggacggcggcaccgagcccaacgacaccgagaccttcaggcccggcggcggcgacatgaggaacaactggaggagcgagctgtacaagtacaaggtggtggagatcaagcccctgggcgtggcccccaccaccaccaagaggagggtggtggagagggagaagagggccgtgggcatcggcgccgtgttcctgggcttcctgggcgtggccggcagcaccatgggcgccgccagcatcaccctgaccgtgcaggccaggcagctgctgagcggcatcgtgcagcagcagagcaacctgctgagggccatcgaggcccagcagcacctgctgcagctgaccgtgtggggcatcaagcagctgcagaccagggtgctggccatcgagaggtacctgaaggaccagcagctgctgggcatctggggctgcagcggcaagctgatctgcaccaccgccgtgccctggaacagcagctggagcaacaagagccagaaggagatctgggacaacatgacctggatgcagtgggacaaggagatcagcaactacaccaacaccgtgtacaggctgctggaggagagccagaaccagcaggagaggaacgagaaggacctgctggccctggacagctggaagaacctgtggagctggttcgacatcaccaactggctgtggtacatcaagatcttcatcatcatcgtgggcggcctgatcggcctgaggatcatcttcgccgtgctgagcatcgtgaacagggtgaggcagggctactga

SEQ ID NO: 2 - DNA, 1488 bp, HIV-1
atgggtgcgagagcgtcaatattaagagggggaaaattagataaatgggaaaaaattaggttaaggccagggggaaagaaacactatatgctaaaacacctagtatgggcaagctgggagctggaaagatttgcacttaaccctggccttttagagacatcagaaggctgtaaacaaataatgaaacagctacaaccagctcttcagacaggaacagaggaacttagatcattattcaacacagtagcaactctctattgtgtacatacagagatagatgtacgagacaccaaagaagccttagacaagatagaggaagaacaaaacaaaattcagcaaaaaacacagcaggcaaaggaggctgacgggaaggtcagtcaaaattatcctatagtacagaatctccaagggcaaatggtacatcagcccatatcacctagaactttaaatgcatgggtaaaagcggtagaagagaaggcttttagcccagaagtaatacccatgttttcagcgttatcagaaggagccaccccacaagatttaaacaccatgctaaacacagtggggggacatcaagcagccatgcaaatattaaaagataccatcaatgaagaggctgcagaatgggatagattacatccagtacatgcagggcctattgcaccaggccaaatgagagaaccaaggggaagtgacatagcaggaactactagtaacctacaggaacaaatagcatggatgacgagtaacccacctgttccagtaggagacatctataaaagatggataattctgggattaaataaaatagtaagaatgtatagccctaccagcattctggacataaaacaagggccaaaggaaccctttagagactatgtagaccggttctttaaaactttaagagcggaacaagctacacaagatgtaaaaaattggatgacagacaccttgttggtccaaaatgcgaacccagattgtaagaccattttaagagcattaggaccaggggcttcaatagaagaaatgatgacagcatgtcagggagtgggaggacctagccataaagcaaaagtgttggccgaggcaatgagccaaacaaacagtgccatactgatgcagagaagcaattttaaaggctctaaaagaattgttaaatgtttcaactgtggcaagggagggcacatagccagaaattgcagggcccctaggaaaaagggctgttggaaatgtggaaaagaaggacaccaaatgaaagattgtactgagagacaggccaattttttagggaaaatctggccctcccacaagggaaggccagggaattttcttcagaacagaccagagccaacagccccaccagaggagagcttcaggtttggggaagagacaacaactccatctcagaagcaggagccaatagacaaggaactatatcctttaacttccctcaaatcactctttggcaacgacccctcgtcacaataa

SEQ ID NO: 3 - DNA, 1154 bp, HSV-1
atggcttcgtacccctgccatcagcacgcgtctgcgttcgaccaggctgcgcgttctcgcggcaatagcaaccgacgtacggcgttgcgccctcgccggcagcaagaagccacggaagtccgcccggagcagataatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaaccaccaccaccaccaccactgaaagct

SEQ ID NO: 4 - DNA, 6450 bp, Artificial Sequence
  Artificially synthesized plasmid pSC65-DR-GN
agcttttgggatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgtcaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagttacggaacgggactatggacgcatgataagaataattttgaagcattggaagcaactaaactatgtgatctcttggaatcaattacagatttctccgtgataggtatcgatgaaggacagttctttccagacattgttgaattagatcgataaaaattaattaattacccgggtaccaggcctagtactgtcgacttcgagcttatttatattccaaaaaaaaaaaataaaatttcaatttttaagctttacatcattccaaacccacccgctttttatagtaagtttttcacccataaataataaatacaataattaatttctcgtaaaagtagaaaatatattctaatttattgcacggtaaggaagtagatcataactcgagtctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtaaaaagtgatacatcgactcataatgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgccaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagcttgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagcgaattcggtctggcgagaccattacccacatgataagagattgtatcagtttcgtagtcttgagtattggtattactatatagtatatagacgtctagaggatcgatccccagattacaaacaactaggaaattggtttataatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatagatcgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgacggatccttctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctactaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcag

SEQ ID NO: 5 - DNA, 622 bp, Vaccinia virus
atcgttgacatctagtattgatagatggaagccatcaaaaccatattggcagaagtatgctaaaatgcgcgaaccaaaatgtgatatgggggttgcgatgttaaacggattaatatatgtcatgggtggaatcgttaaaggtgacacgtgtaccgacgcactagagagtttatcagaagatggatggatgaagcatcaacgtcttccaataaaaatgtccaatatgtcgacgattgttcatgatggcaagatttatatatctggaggttacaacaatagtagtgtagttaatgtaatatcgaatctagtccttagctataatccgatatatgatgaatggaccaaattatcatcattaaatattcctagaattaatcccgctctatggtcagcgcataataaattatatgtaggaggaggaatatctgatgatgttcgaactaatacatctgagacatacgacaaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatag

SEQ ID NO: 6 - DNA, 587 bp, Vaccinia virus
tgacttacataaatgtctgggatagtaaaatctatcatattgagcggaccatctggttcaggaaagacagccatagccaaaagactatgggaatatatttggatttgtggtgtcccataccactagatttcctcgtcctatggaacgagaaggtgtcgattaccattacgttaacagagaggccatctggaagggaatagccgccggaaactttctagaacatactgagtttttaggaaatatttacggaacttctaaaacagctgtgaatacagcggctattaataatcgtatttgtgtgatggatctaaacatcgacggtgttagaagttttaaaaatacttacctaatgccttactcggtgtatataagacctacctctcttaaaatggttgagaccaagcttcgttgtagaaacactgaagcggatgatgagattcatcgtcgtgtgatgttggcaaaaactgacatggatgaggcaggtgaagccggtctattcgacactattattattgaagatgatgtgaatttagcatatagtaagttaattcagatactacaggaccgtattagaatgtattttaaca

SEQ ID NO: 7 - DNA, 6188 bp, Artificial Sequence
  Artificially synthesized plasmid pLW73-Neo-I8RDR
gaattccctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctctcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggaattatcccgatctatacgatacaaacttaacggatatcgcgataatgaaataatttatgattatttctcgctttcaatttaacacaaccctcaagaacctttgtatttattttcattttttaagtatagaataaagaatctataaaaactaaaaaaattatacattataaaccaatttcctagttgtttgtaatctggggatcgatcctctagacatctatatactatatagtaataccaatactcaagactacgaaactgatacaatctcttatcatgtgggtaatggtctcgccagaccaactggtaagatatcgagctcgaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaactcgaggccgctggtacccaacctaaaaattgaaaataaatacaaaggttcttgagggttgtgttaaattgaaagcgagaaataatcataaataagcccggggatcctctagagtcgacctgcagtcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaacaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 8 - DNA, 260 bp, Vaccinia virus
agaattaatcccgctctatggtcagcgcataataaattatatgtaggaggaggaatatctgatgatgttcgaactaatacatctgagacatacgacaaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagaacaa

SEQ ID NO: 9 - DNA, 6117 bp, Artificial Sequence
  Artificially synthesized plasmid pVI76
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatcgttgacatctagtattgatagatggaagccatcaaaaccatattggcagaagtatgctaaaatgcgcgaaccaaaatgtgatatgggggttgcgatgttaaacggattaatatatgtcatgggtggaatcgttaaaggtgacacgtgtaccgacgcactagagagtttatcagaagatggatggatgaagcatcaacgtcttccaataaaaatgtccaatatgtcgacgattgttcatgatggcaagatttatatatctggaggttacaacaatagtagtgtagttaatgtaatatcgaatctagtccttagctataatccgatatatgatgaatggaccaaattatcatcattaaatattcctagaattaatcccgctctatggtcagcgcataataaattatatgtaggaggaggaatatctgatgatgttcgaactaatacatctgagacatacgacaaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctctcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggaattatcccgatctatacgatacaaacttaacggatatcgcgataatgaaataatttatgattatttctcgctttcaatttaacacaaccctcaagaacctttgtatttattttcattttttaagtatagaataaagaatctataaaaactaaaaaaattatacattataaaccaatttcctagttgtttgtaatctggggatcgatcctctagacatctatatactatatagtaataccaatactcaagactacgaaactgatacaatctcttatcatgtgggtaatggtctcgccagaccaactggtaagatatcgagctcgagagaattaatcccgctctatggtcagcgcataataaattatatgtaggaggaggaatatctgatgatgttcgaactaatacatctgagacatacgacaaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagaacaagtcgtccgcaagcttaaaaattgaaattttattttttttttttggaatataaataagctcgaagtcgacagtactaggcctggtacccgggtaattaattaagacttacataaatgtctgggatagtaaaatctatcatattgagcggaccatctggttcaggaaagacagccatagccaaaagactatgggaatatatttggatttgtggtgtcccataccactagatttcctcgtcctatggaacgagaaggtgtcgattaccattacgttaacagagaggccatctggaagggaatagccgccggaaactttctagaacatactgagtttttaggaaatatttacggaacttctaaaacagctgtgaatacagcggctattaataatcgtatttgtgtgatggatctaaacatcgacggtgttagaagttttaaaaatacttacctaatgccttactcggtgtatataagacctacctctcttaaaatggttgagaccaagcttcgttgtagaaacactgaagcggatgatgagattcatcgtcgtgtgatgttggcaaaaactgacatggatgaggcaggtgaagccggtctattcgacactattattattgaagatgatgtgaatttagcatatagtaagttaattcagatactacaggaccgtattaaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 49 bp, Artificial Sequence
  Artificially synthesized oligonucleotide primer
caagaattcctcgagtcagtggtggtggtggtggtggttagcctccccc

SEQ ID NO: 11 - DNA, 34 bp, Artificial Sequence
  Artificially synthesized oligonucleotide primer
tacgtcgacatggcttcgtacccctgccatcagc

SEQ ID NO: 12 - DNA, 35 bp, Artificial Sequence
  Artificially synthesized oligonucleotide primer
acgcgtcgacagagatatcgacaccatggacaggg

SEQ ID NO: 13 - DNA, 36 bp, Artificial Sequence
  Artificially synthesized oligonucleotide primer
gccttaattaatcagtagccctgcctcaccctgttc

SEQ ID NO: 14 - DNA, 25 bp, Artificial Sequence
  Artificially synthesized oligonucleotide primer
taacgtgatggatatattaaagtcg

SEQ ID NO: 15 - DNA, 24 bp, Artificial Sequence
  Artificially synthesized oligonucleotide primer
aacgacacaacatccatttttaag

SEQ ID NO: 16 - DNA, 42 bp, Artificial Sequence
  Artificially synthesized oligonucleotide primer
catacgcgatcagaattcatcgttgacatctagtattgatag

SEQ ID NO: 17 - DNA, 46 bp, Artificial Sequence
  Artificially synthesized oligonucleotide primer
taaggcctctcgaggcgcgccctatcaactacctataaaactttcc

SEQ ID NO: 18 - DNA, 45 bp, Artificial Sequence
  Artificially synthesized oligonucleotide primer
gagaacctcgagttaattaatgacttacataaatgtctgggatag

SEQ ID NO: 19 - DNA, 34 bp, Artificial Sequence
  Artificially synthesized oligonucleotide primer
tctaggccttgttaaaatacattctaatacggtc

SEQ ID NO: 20 - DNA, 35 bp, Artificial Sequence
  Artificially synthesized oligonucleotide primer
aaatctcgagagaattaatcccgctctatggtcag

SEQ ID NO: 21 - DNA, 34 bp, Artificial Sequence
  Artificially synthesized oligonucleotide primer
gcgaagcttttgttctatcaactacctataaaac

SEQ ID NO: 22 - DNA, 25 bp, Artificial Sequence
  Artificially synthesized oligonucleotide primer
tgttaaaatacattctaatacggtc

SEQ ID NO: 23 - DNA, 24 bp, Artificial Sequence
  Artificially synthesized oligonucleotide primer
atcgttgacatctagtattgatag